(19) United States
(12) Patent Application Publication
(10) Pub. No.: US 2010/0211030 A1
(43) Pub. Date: Aug. 19, 2010

(54) WOUND TREATMENT APPARATUS WITH A CONTROL SYSTEM CONNECTED TO A FLOW METER AND A PRESSURE SENSOR

(75) Inventors: Jake Turner, Cambridge (GB); Benjamin Gordon, Cambridge (GB)

Correspondence Address: KNOBBE MARTENS OLSON & BEAR LLP, 2040 MAIN STREET, FOURTEENTH FLOOR, IRVINE, CA, 92614, US

(73) Assignee: SMITH & NEPHEW PLC, London (GB)

(21) Appl. No.: 12/667,228

(22) PCT Filed: Jun. 27, 2008
(86) PCT No.: PCT/GB2008/050507

§ 371 Date: Dec. 29, 2009

(30) Foreign Application Priority Data
Jul. 2, 2007 (GB) 0712736.8

(51) Int. Cl. A61M 1/00; G08B 21/00

(52) U.S. Cl. 604/319; 340/612

(57) ABSTRACT

Apparatus and a method are described for the provision of topical negative pressure therapy, the apparatus comprising: an aspirant pump for moving aspirated fluid through said apparatus; aspirant conduit means operably connected to a dressing covering a wound being aspirated; a waste container operably connected to the aspirant conduit and for receiving wound exudate therein; the waste container having a fluid exit port for the flow of gaseous aspirated fluid therefrom, the exit port having filter means associated therewith for preventing aspirated liquid from passing therethrough; a fluid flow path on the exit side of said waste container for the flow of aspirated gaseous fluid therethrough;

the fluid flow path having therein a flow meter and a pressure monitor; and a control system for interrogating and interpreting signals from said flow meter and pressure monitor. The apparatus and method alerts a user to an approaching waste canister full condition.

The present invention relates to apparatus and a method for the application of topical negative pressure (TNP) therapy to wounds. In particular, but not exclusively, the present invention relates to alerting a user of such apparatus to a canister which is full or approaching full.

There is much prior art available relating to the provision of apparatus and methods of use thereof for the application of TNP therapy to wounds together with other therapeutic processes intended to enhance the effects of the TNP therapy. Examples of such prior art include those listed and briefly described below.

TNP therapy assists in the closure and healing of wounds by reducing tissue oedema; encouraging blood flow and granulation of tissue; removing excess exudates and may reduce bacterial load and thus, infection to the wound. Furthermore, TNP therapy permits less outside disturbance of the wound and promotes more rapid healing.

In our co-pending International patent application, WO 2004/037334, apparatus, a wound dressing and a method for aspirating, irrigating and cleansing wounds are described. In very general terms, this invention describes the treatment of a wound by the application of topical negative pressure (TNP) therapy for aspirating the wound together with the further provision of additional fluid for irrigating and/or cleansing the wound, which fluid, comprising both wound exudates and irrigation fluid, is then drawn off by the aspiration means and circulated through means for separating the beneficial materials therein from deleterious materials. The materials which are beneficial to wound healing are recirculated through the wound dressing and those materials deleterious to wound healing are discarded to a waste collection bag or vessel.

In our co-pending International patent application, WO 2005/04670, apparatus, a wound dressing and a method for cleansing a wound using aspiration, irrigation and cleansing wounds are described. Again, in very general terms, the invention described in this document utilises similar apparatus to that in WO 2004/037334 with regard to the aspiration, irrigation and cleansing of the wound, however, it further includes the important additional step of providing heating means to control the temperature of that beneficial material being returned to the wound site/dressing so that it is at an optimum temperature, for example, to have the most efficacious therapeutic effect on the wound.

In our co-pending International patent application, WO 2005/105180, apparatus and a method for the aspiration, irrigation and/or cleansing of wounds are described. Again, in very general terms, this document describes similar apparatus to the two previously mentioned documents hereinabove but with the additional step of providing means for the supply and application of physiologically active agents to the wound site/dressing to promote wound healing.

The content of the above references is included herein by reference.

However, the above apparatus and methods are generally only applicable to a patient when hospitalised as the apparatus is complex, needing people having specialist knowledge in how to operate and maintain the apparatus, and also relatively heavy and bulky, not being adapted for easy mobility outside of a hospital environment by a patient, for example.

Some patients having relatively less severe wounds which do not require continuous hospitalisation, for example, but whom nevertheless would benefit from the prolonged application of TNP therapy, could be treated at home or at work subject to the availability of an easily portable and maintainable TNP therapy apparatus.

It is an aim of embodiments of the present invention to provide an apparatus and method for alerting a user or wearer of the apparatus that the waste canister is full or approaching the full condition and that a new canister should be checked and/or possibly installed.

According to a first aspect of the present invention there is provided apparatus for the provision of topical negative pressure therapy the apparatus comprising: an aspirant pump for moving aspirated fluid through said apparatus; aspirant conduit means operably connected to a dressing covering a wound being aspirated; a waste container operably connected to the aspirant conduit and for receiving wound exudate therein; the waste container having a fluid exit port for the flow of gaseous aspirated fluid therefrom, the exit port having filter means associated therewith for preventing aspirated liquid from passing therethrough; a fluid flow path on the exit side of said waste container for the flow of aspirated gaseous fluid therethrough; the fluid flow path having therein a flow meter and a pressure monitor; and a control system for interrogating and interpreting signals from said flow meter and pressure monitor.

The invention is comprised in part of an overall apparatus for the provision of TNP therapy to a patient in almost any environment. The apparatus is lightweight, may be mains or battery powered by a rechargeable battery pack contained within a device (henceforth, the term “device” is used to connote a unit which, may contain all of the control, power supply, power supply recharging, electronic indicator means and means for initiating and sustaining aspiration functions to a wound and any further necessary functions of a similar nature). When outside the home, for example, the apparatus may provide for an extended period of operation on battery power and in the home, for example, the device may be connected to the mains by a charger unit whilst still being used and operated by the patient.

The overall apparatus of which the present invention is a part comprises: a dressing covering the wound and sealing at least an open end of an aspiration conduit to a cavity formed over the wound by the dressing; an aspiration tube comprising at least one lumen therethrough leading from the wound dressing to a waste material canister for collecting and holding wound exudates/waste material prior to disposal; and, a power, control and aspiration initiating and sustaining device associated with the waste canister.

The dressing covering the wound may be any type of dressing normally employed with TNP therapy and, in very general terms, may comprise, for example, a semi-permeable, flexible, self-adhesive drape material, as is known in the dressings art, to cover the wound and seal with surrounding sound tissue to create a sealed cavity or void over the wound. There may aptly be a porous barrier and support member in the cavity between the wound bed and the covering material to enable an even vacuum distribution to be achieved over the area of the wound. The porous barrier and support member being, for example, a gauze, a foam, an inflatable bag or known wound contact type material resistant to crushing under the levels of vacuum created and which permits transfer of wound exudates across the wound area to the aspiration conduit sealed to the flexible cover drape over the wound.

The aspiration conduit may be a plain flexible tube, for example, having a single lumen therethrough and made from a plastics material compatible with raw tissue, for example. However, the aspiration conduit may have a plurality of lumens therethrough to achieve specific objectives relating to the invention. A portion of the tube sited within the sealed cavity over the wound may have a structure to enable continued aspiration and evacuation of wound exudates without becoming constricted or blocked even at the higher levels of the negative pressure range envisaged.

It is envisaged that the negative pressure range for the apparatus embodying the present invention may be between about −50 mmHg and −200 mmHg (note that these pressures are relative to normal ambient atmospheric pressure thus, −200 mmHg would be about 560 mmHg in practical terms). Aptly, the pressure range may be between about −75 mmHg and −150 mmHg. Alternatively a pressure range of up to −75 mmHg, up to −80 mmHg or over −80 mmHg can be used. Also aptly a pressure range of below −75 mmHg could be used. Alternatively a pressure range of over −100 mmHg could be used or over −150 mmHg.

The aspiration conduit at its distal end remote from the dressing may be attached to the waste canister at an inlet port or connector. The device containing the means for initiating and sustaining aspiration of the wound/dressing may be situated between the dressing and waste canister, however, in a preferred embodiment of the apparatus embodying the present invention, the device may aspirate the wound/dressing via the canister thus, the waste canister may preferably be sited between the wound/dressing and device.

The aspiration conduit at the waste material canister end may preferably be bonded to the waste canister to prevent inadvertent detachment when being caught on an obstruction, for example.

The canister may be a plastics material moulding or a composite unit comprising a plurality of separate mouldings. The canister may aptly be translucent or transparent in order to visually determine the extent of filling with exudates. However, the canister and device may in some embodiments provide automatic warning of imminent canister full condition and may also provide means for cessation of aspiration when the canister reaches the full condition.

The canister is provided with a filter or filters to prevent the exhaust of liquids and odours therefrom and also to prevent the expulsion of bacteria into the atmosphere. Such filters may comprise a plurality of filters in series. Examples of suitable filters may comprise hydrophobic filters of 0.2 μm pore size, for example, in respect of sealing the canister against bacteria expulsion and 1 μm against liquid expulsion.

Aptly, the filters may be sited at an upper portion of the waste canister in normal use, that is when the apparatus is being used or carried by a patient the filters are in an upper position and separated from the exudate liquid in the waste canister by gravity. Furthermore, such an orientation keeps the waste canister outlet or exhaust exit port remote from the exudate surface.

Aptly the waste canister may be filled with an absorbent gel such as ISOLYSEL (trade mark), for example, as an added safeguard against leakage of the canister when full and being changed and disposed of. Added advantages of a gel matrix within the exudate storing volume of the waste canister are that it prevents excessive movement, such as slopping, of the liquid, minimises bacterial growth and minimises odours.

The waste canister may also be provided with suitable means to prevent leakage thereof both when detached from the device unit and also when the aspiration conduit is detached from the wound site/dressing.

The canister may have suitable means to prevent emptying by a user (without tools or damage to the canister) such that a full or otherwise end-of-life canister may only be disposed of with waste fluid still contained.

The device and waste canister may have mutually complementary means for connecting a device unit to a waste canister whereby the aspiration means in the device unit automatically connects to an evacuation port on the waste canister such that there is a continuous aspiration path from the wound site/dressing to an exhaust port on the device.

Aptly, the exhaust port from the fluid path or at a position in the fluid path through the apparatus is provided with filter means to prevent offensive odours from being ejected into the atmosphere.

In general terms the device unit comprises an aspirant pump; means for monitoring pressure applied by the aspirant pump; a flowmeter to monitor fluid flow through the aspirant pump; a control system which controls the aspirant pump in response to signals from sensors such as the pressure monitoring means and the flowmeter, for example, and which control system also controls a power management system with regard to an on-board battery pack and the charging thereof and lastly a user interface system whereby various functions of the device such as pressure level set point, for example, may be adjusted (including stopping and starting of the apparatus) by a user. The device unit may contain all of the above features within a single unified casing.

In view of the fact that the device unit contains the majority of the intrinsic equipment cost therein ideally it will also be able to survive impact, tolerate cleaning in order to be reusable by other patients.

In one embodiment of the present invention, the aspirant pump may be placed in the fluid flow path after the waste canister exit port intermediate the aspirant pressure monitor and the aspirant flow meter.

The flow meter may be hot wire or pressure differential or any other suitable flow meter known to those skilled in the art.

A software element of the control system uses the aspirant pressure monitor and the aspirant flow meter to determine the status of the waste canister with respect to its degree of filling and need for changing the waste canister for fresh unit. In essence the software queries the pressure monitor and flow meter at regular intervals when the apparatus is in use to determine the pressure being delivered or applied and the flow of exudate from the wound to the waste canister. If the software detects sufficient pressure (being created by the aspirant pump) but detects an inappropriately low flow rate of gaseous aspirant fluid for the detected pressure, the software will classify the condition as being either a blockage in the aspirant conduit, or of the filter at the waste canister exit port or, a full waste canister. Under this condition the software alerts the user by means of an alarm such as a buzzer, a flashing light or a message on the device LCD screen or a combination of some or all of these, for example. The user may then check the waste canister and take the appropriate action.

The alarm system may include at least one of; an audible alarm; a visible alarm; a physically conveyed alarm such as a vibration; and a message alarm on a LCD screen.

Even in the condition where an alarm has been raised, the aspirant pump may still be kept running in order to continue the TNR therapy since the alarm may be caused by energetic movement of the user such as by running, for example and the filter at the exit in the waste canister may be only momentarily or temporarily blocked.

The fluid flow path may be provided with a silencing system to reduce noise generated by the aspirant pump and may also have a filter on an exhaust port to atmosphere, for example. In the embodiment of the present invention described above the silencing system and the exhaust filter may be positioned in the fluid flow path downstream of the pressure monitor, the flow meter and the aspirant pump.

Other embodiments of the apparatus according to the present invention may position the elements of the system in a different order with respect to the flow path. Positional variations starting from the waste canister exit port may include: pressure monitor, aspirant flowmeter, aspirant pump; or, pressure monitor, aspirant pump, silencing system, flow meter, exhaust filter; or, flow meter, pressure monitor, aspirant pump, silencing system, exhaust filter.

In terms of pressure capability the aspiration means may be able to apply a maximum pressure drop of at least −200 mmHg to a wound site/dressing. The apparatus is capable of maintaining a predetermined negative pressure even under conditions where there is a small leak of air into the system and a high exudate flow.

The pressure control system may prevent the minimum pressure achieved from exceeding for example −200 mmHg so as not to cause undue patient discomfort. The pressure required may be set by the user at a number of discreet levels such as −50, −75, −100, −125, −150, −175 mmHg, for example, depending upon the needs of the wound in question and the advice of a clinician. Thus suitable pressure ranges in use may be from −25 to −80 mmHg, or −50 to −76 mmHg, or −50 to −75 mmHg as examples. The control system may also advantageously be able to maintain the set pressure within a tolerance band of +/−10 mmHg of the set point for 95% of the time the apparatus is operating given that leakage and exudation rates are within expected or normal levels.

Aptly, the control system may trigger alarm means such as a flashing light, buzzer or any other suitable means when various abnormal conditions apply such as, for example: pressure outside set value by a large amount due to a gross leak of air into system; duty on the aspiration pump too high due to a relatively smaller leakage of air into the system; pressure differential between wound site and pump is too high due, for example, to a blockage or waste canister full.

The apparatus of the present invention may be provided with a carry case and suitable support means such as a shoulder strap or harness, for example. The carry case may be adapted to conform to the shape of the apparatus comprised in the joined together device and waste canister. In particular, the carry case may be provided with a bottom opening flap to permit the waste canister to be changed without complete removal of the apparatus form the carry case.

The carry case may be provided with an aperture covered by a displaceable flap to enable user access to a keypad for varying the therapy applied by the apparatus.

According to a second aspect of the present invention, there is provided a method for determining a waste canister full condition of apparatus for the topical negative pressure treatment of a wound comprising the steps of: providing an aspirant pump for moving aspirated fluid through said apparatus; providing aspirant conduit means operably connected to a dressing covering a wound being aspirated; providing a waste container operably connected to the aspirant conduit and for receiving wound exudate therein;

providing the waste container with a fluid exit port for the flow of gaseous aspirated fluid therefrom, the exit port having filter means associated therewith for preventing aspirated liquid from passing therethrough; providing a fluid flow path on the exit side of said waste container for the flow of aspirated gaseous fluid therethrough; providing the fluid flow path with a flow meter and a pressure monitor; and a control system for interrogating and interpreting signals from said flow meter and pressure monitor.

According to a third aspect of the present invention there is provided a method for the use of apparatus according to the first aspect for the provision of topical negative pressure therapy to a user.

In order that the present invention may be more fully understood, examples will now be described by way of illustration only with reference to the accompanying drawings, of which:

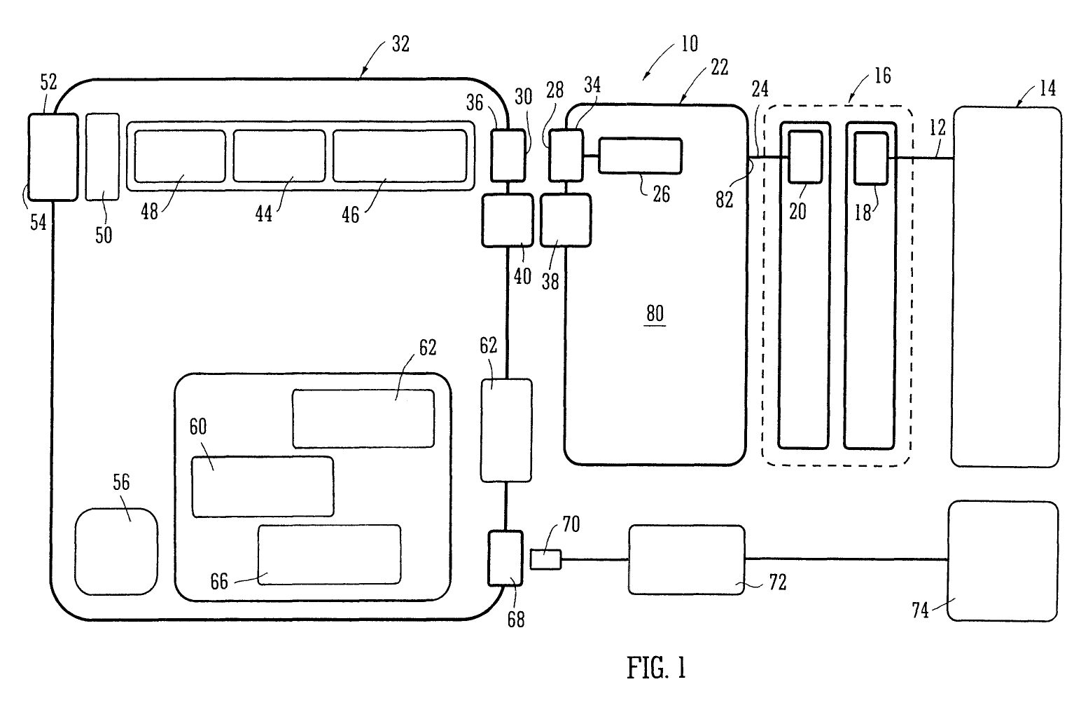

FIG. 1 shows a generalised schematic block diagram showing a general view of an apparatus and the constituent apparatus features thereof;

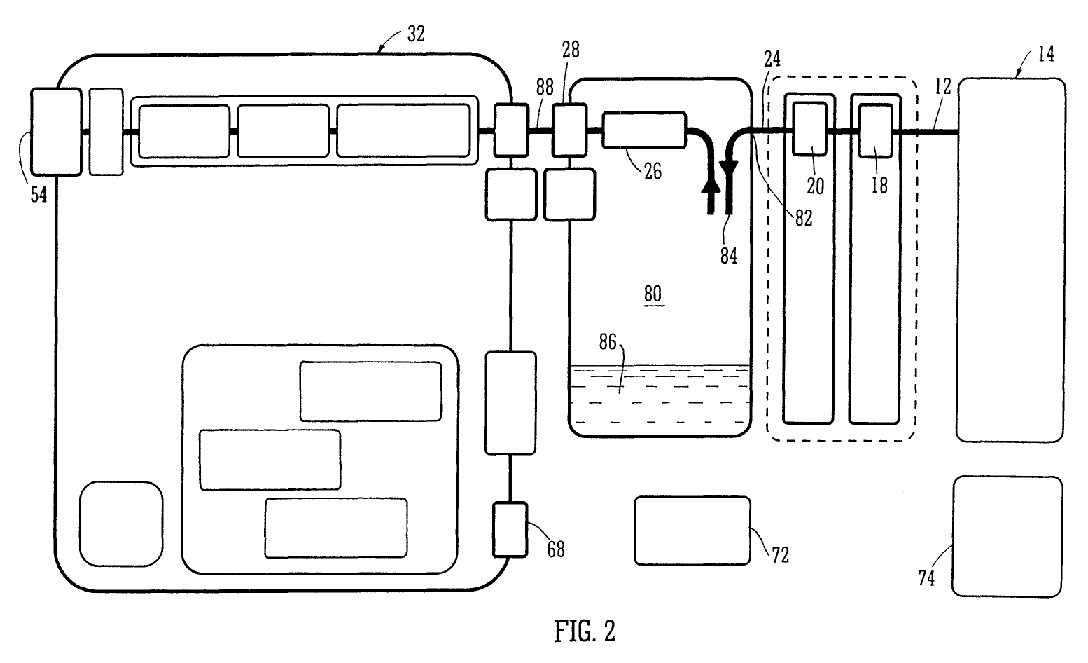

FIG. 2 shows a similar generalised schematic block diagram to FIG. 1 and showing fluid paths therein;

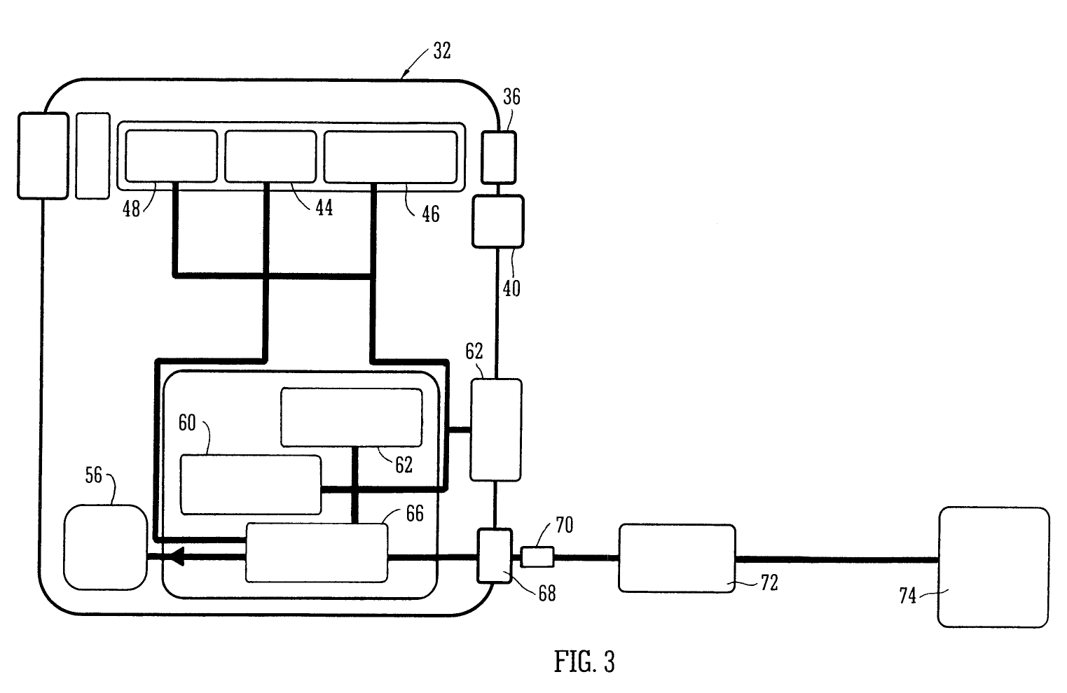

FIG. 3 shows a generalised schematic block diagram similar to FIG. 1 but of a device unit only and showing power paths for the various power consuming/producing features of the apparatus;

FIG. 4 shows a similar generalised schematic block diagram to FIG. 3 of the device unit and showing control system data paths for controlling the various functions and components of the apparatus;

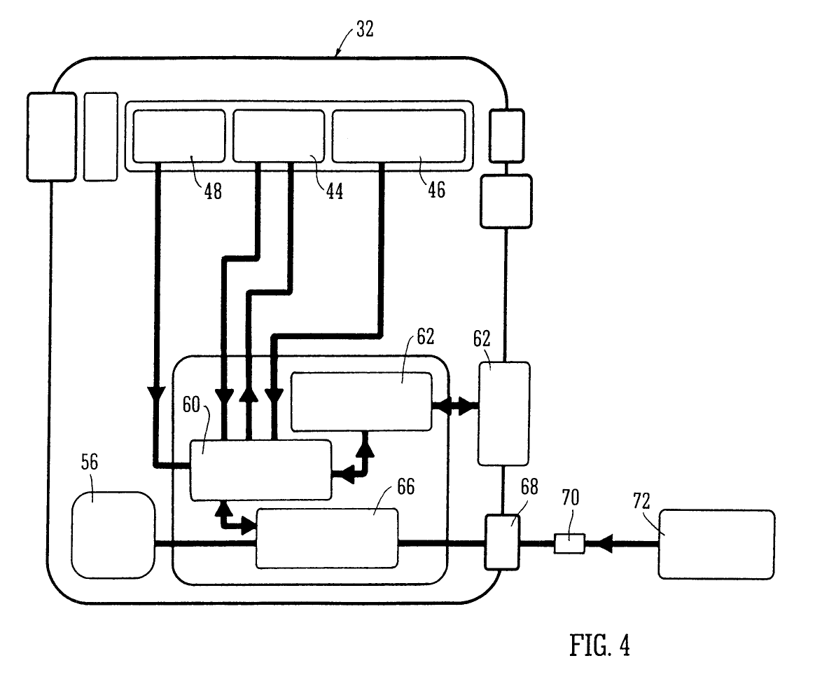

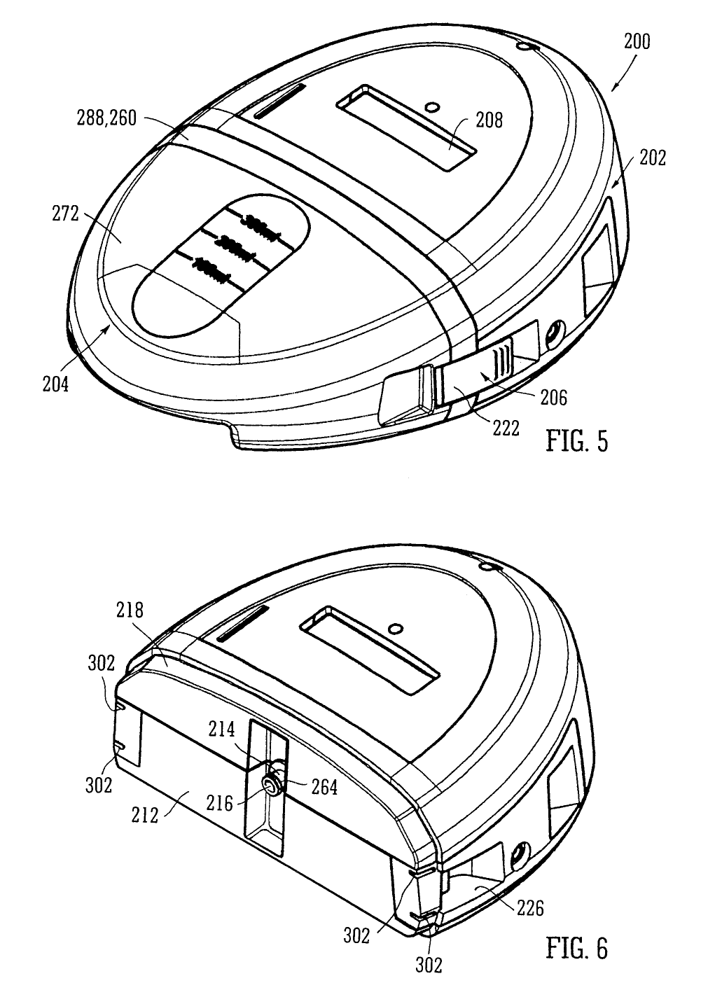

FIG. 5 shows a perspective view of an apparatus;

FIG. 6 shows a perspective view of an assembled device unit of the apparatus of FIG. 5;

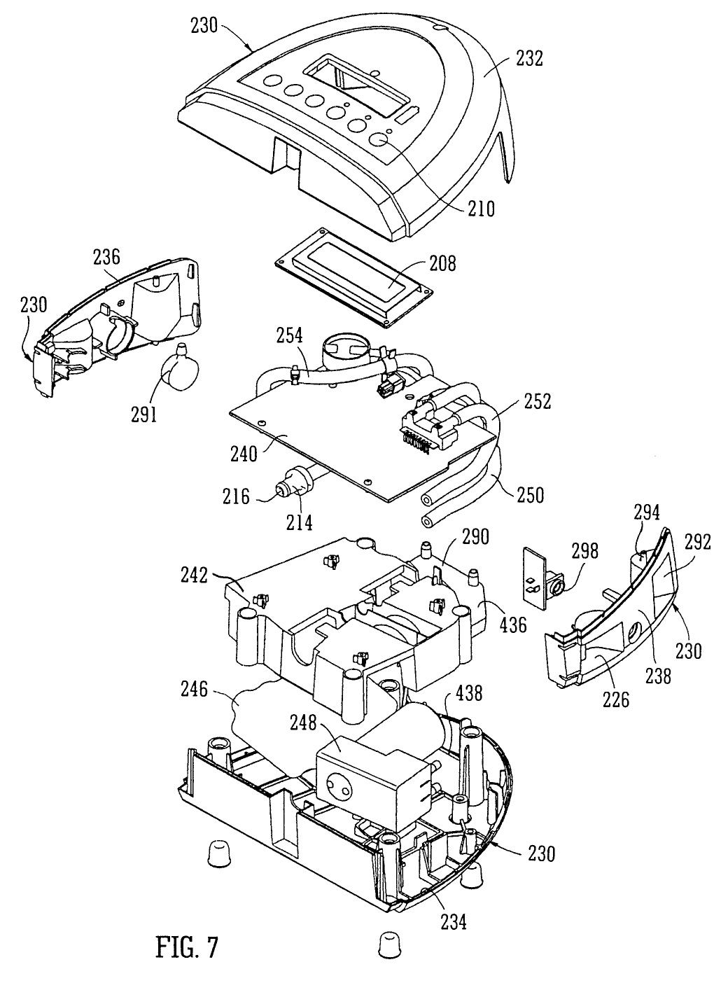

FIG. 7 shows an exploded view of the device unit of FIG. 6;

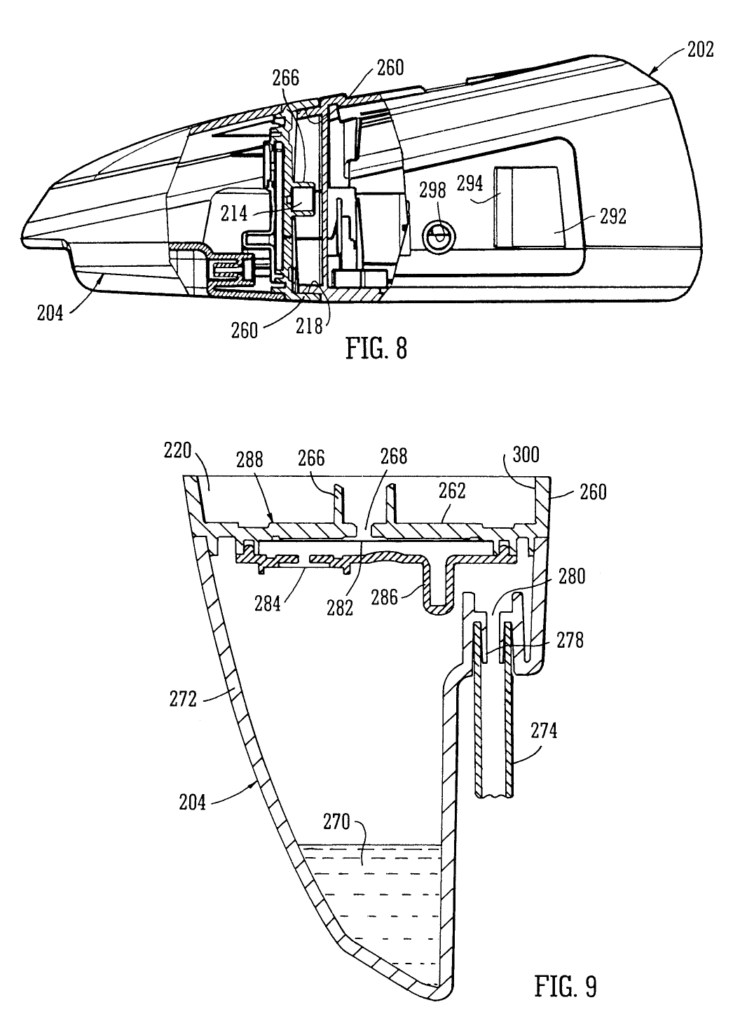

FIG. 8 shows a partially sectioned side elevation view through the interface between a waste canister and device unit of the apparatus;

FIG. 9 shows a cross section through a waste canister of the apparatus of FIGS. 5 to 8;

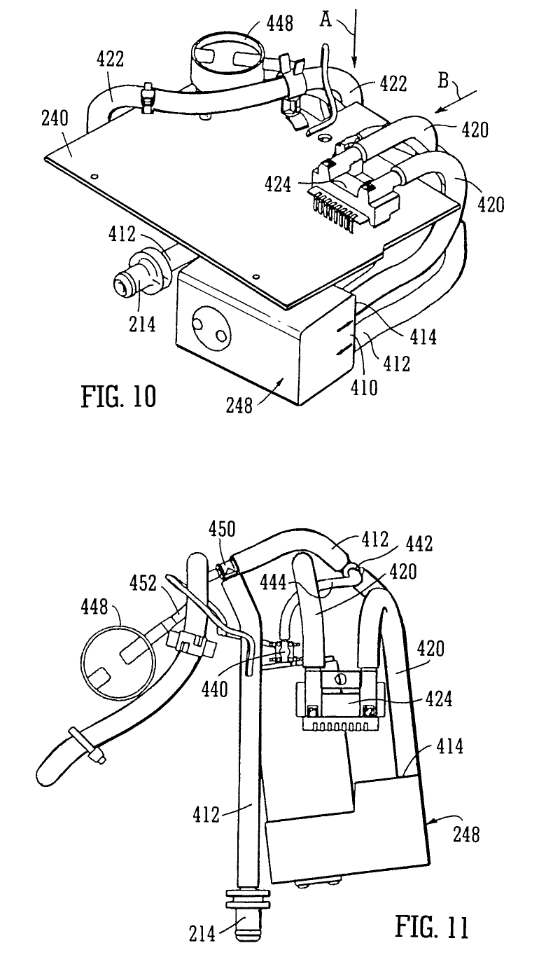

FIG. 10 shows a detail of a preferred embodiment of apparatus for the application TNP therapy and embodying functional elements of a control system for determining a waste canister full condition according to the present invention;

FIG. 11 which is a view of the apparatus of FIG. 11 in the direction of arrow A of FIG. 10 but with chassis plate omitted; and

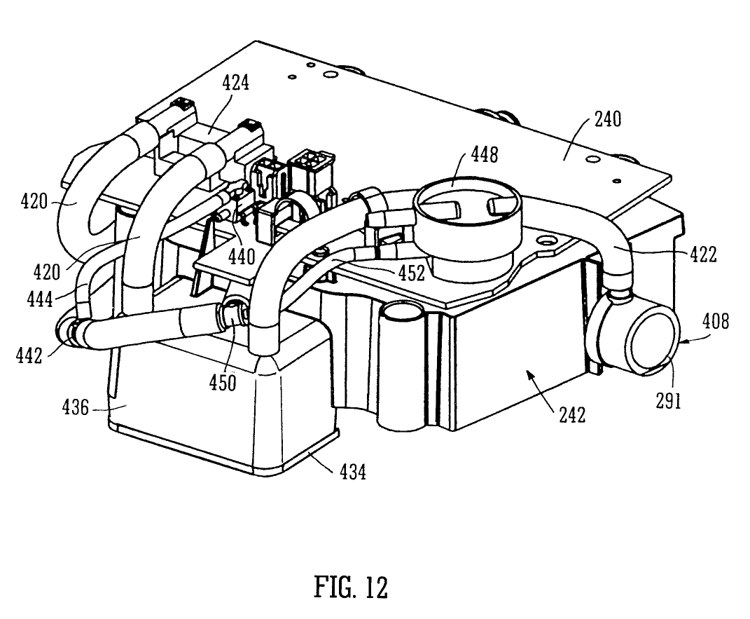

FIG. 12 which is a view of the apparatus of FIG. 10 in the direction of arrow B of FIG. 10.

Referring now to FIGS. 1 to 4 of the drawings and where the same or similar features are denoted by common reference numerals.

FIG. 1 shows a generalised schematic view of an apparatus 10 of a portable topical negative pressure (TNP) system. It will be understood that embodiments of the present invention are generally applicable to use in such a TNP system. Briefly, negative pressure wound therapy assists in the closure and healing of many forms of “hard to heal” wounds by reducing tissue oedema; encouraging blood flow and granular tissue formation; removing excess exudate and may reduce bacterial load (and, therefore, infection). In addition the therapy allows for less disturbance of a wound leading to more rapid healing. The TNP system is detailed further hereinafter but in summary includes a portable body including a canister and a device with the device capable of providing an extended period of continuous therapy within at least a one year life span. The system is connected to a patient via a length of tubing with an end of the tubing operably secured to a wound dressing on the patient.

More particularly, as shown in FIG. 1, the apparatus comprises an aspiration conduit 12 operably and an outer surface thereof at one end sealingly attached to a dressing 14. The dressing 14 will not be further described here other than to say that it is formed in a known manner from well known materials to those skilled in the dressings art to create a sealed cavity over and around a wound to be treated by TNP therapy with the apparatus of the present invention. The aspiration conduit has an in-line connector 16 comprising connector portions 18, 20 intermediate its length between the dressing 14 and a waste canister 22. The aspiration conduit between the connector portion 20 and the canister 22 is denoted by a different reference numeral 24 although the fluid path through conduit portions 12 and 24 to the waste canister is continuous. The connector portions 18, 20 join conduit portions 12, 24 in a leak-free but disconnectable manner. The waste canister 22 is provided with filters 26 which prevent the escape via an exit port 28 of liquid and bacteria from the waste canister. The filters may comprise a 1 μm hydrophobic liquid filter and a 0.2 μm bacteria filter such that all liquid and bacteria is confined to an interior waste collecting volume of the waste canister 22. The exit port 28 of the waste canister 22 mates with an entry/suction port 30 of a device unit 32 by means of mutually sealing connector portions 34, 36 which engage and seal together automatically when the waste canister 22 is attached to the device unit 32, the waste canister 22 and device unit 32 being held together by catch assemblies 38, 40. The device unit 32 comprises an aspirant pump 44, an aspirant pressure monitor 46 and an aspirant flowmeter 48 operably connected together. The aspiration path takes the aspirated fluid which in the case of fluid on the exit side of exit port 28 is gaseous through a silencer system 50 and a final filter 52 having an activated charcoal matrix which ensures that no odours escape with the gas exhausted from the device 32 via an exhaust port 54. The filter 52 material also serves as noise reducing material to enhance the effect of the silencer system 50. The device 32 also contains a battery pack 56 to power the apparatus which battery pack also powers the control system 60 which controls a user interface system 62 controlled via a keypad (not shown) and the aspiration pump 44 via signals from sensors 46, 48. A power management system 66 is also provided which controls power from the battery pack 56, the recharging thereof and the power requirements of the aspirant pump 44 and other electrically operated components. An electrical connector 68 is provided to receive a power input jack 70 from a SELV power supply 72 connected to a mains supply 74 when the user of the apparatus or the apparatus itself is adjacent a convenient mains power socket.

FIG. 2 shows a similar schematic representation to FIG. 1 but shows the fluid paths in more detail. The wound exudate is aspirated from the wound site/dressing 14 via the conduit 12, the two connector portions 18, 20 and the conduit 24 into the waste canister 22. The waste canister 22 comprises a relatively large volume 80 in the region of 500 ml into which exudate from the wound is drawn by the aspiration system at an entry port 82. The fluid 84 drawn into the canister volume 80 is a mixture of both air drawn into the dressing 14 via the semi-permeable adhesive sealing drape (not shown) and liquid 86 in the form of wound exudates. The volume 80 within the canister is also at a lowered pressure and the gaseous element 88 of the aspirated fluids is exhausted from the canister volume 80 via the filters 26 and the waste canister exhaust exit port 28 as bacteria-free gas. From the exit port 28 of the waste canister to the final exhaust port 54 the fluid is gaseous only.

FIG. 3 shows a schematic diagram showing only the device portion of the apparatus and the power paths in the device of the apparatus embodying the present invention. Power is provided mainly by the battery pack 56 when the user is outside their home or workplace, for example, however, power may also be provided by an external mains 74 supplied charging unit 72 which when connected to the device 32 by the socket 68 is capable of both operating the device and recharging the battery pack 56 simultaneously. The power management system 66 is included so as to be able to control power of the TNP system. The TNP system is a rechargeable, battery powered system but is capable of being run directly from mains electricity as will be described hereinafter more fully with respect to the further figures. If disconnected from the mains the battery has enough stored charge for approximately 8 hours of use in normal conditions. It will be appreciated that batteries having other associated life times between recharge can be utilised. For example batteries providing less than 8 hours or greater than 8 hours can be used. When connected to the mains the device will run off the mains power and will simultaneously recharge the battery if depleted from portable use. The exact rate of battery recharge will depend on the load on the TNP system. For example, if the wound is very large or there is a significant leak, battery recharge will take longer than if the wound is small and well sealed.

FIG. 4 shows the device 32 part of the apparatus embodying the present invention and the data paths employed in the control system for control of the aspirant pump and other features of the apparatus. A key purpose of the TNP system is to apply negative pressure wound therapy. This is accomplished via the pressure control system which includes the pump and a pump control system. The pump applies negative pressure; the pressure control system gives feedback on the pressure at the pump head to the control system; the pump control varies the pump speed based on the difference between the target pressure and the actual pressure at the pump head. In order to improve accuracy of pump speed and hence provide smoother and more accurate application of the negative pressure at a wound site, the pump is controlled by an auxiliary control system. The pump is from time to time allowed to “free-wheel” during its duty cycle by turning off the voltage applied to it. The spinning motor causes a “back electro-motive force” or BEMF to be generated. This BEMF can be monitored and can be used to provide an accurate measure of pump speed: The speed can thus be adjusted more accurately than can prior art pump systems.

According to embodiments of the present invention, actual pressure at a wound site is not measured but the difference between a measured pressure (at the pump) and the wound pressure is minimised by the use of large filters and large bore tubes wherever practical. If the pressure control measures that the pressure at the pump head is greater than a target pressure (closer to atmospheric pressure) for a period of time, the device sends an alarm and displays a message alerting the user to a potential problem such as a leak.

In addition to pressure control a separate flow control system can be provided. A flow meter may be positioned after the pump and is used to detect when a canister is full or the tube has become blocked. If the flow falls below a certain threshold, the device sounds an alarm and displays a message alerting a user to the potential blockage or full canister.

In more detail and referring again particularly to FIG. 4 a pseudocode for monitoring the condition of filling of the waste canister 22 comprises the sequence of software steps:

| 1) | Check for blockage | | |
|---|---|---|---|
| | i) | Get Pressure value p(current) | |
| | ii) | Get Flow Meter value f(current) | |
| | iii) | If p(current) − p(set) | < allowable pressure difference limit |

| | | |
|---|---|---|
| If f(current) | < | less than min. flow needed |

sound buzzer
display “Blockage/Full” error message

| | |
|---|---|
| 2) | End Check for Blockage. |

Where p(set) is a reference pressure for comparison with the p(current) pressure.

Similarly, f(current) is the instantaneous measured flow rate and should be greater than a preset minimum flow rate under the given pressure conditions.

The above sequence of steps is repeated at a frequency of 200 Hz, however, the sensor may be sampled at a higher frequency and the signals averaged.

The control system 60 obtains the current pressure from the pressure monitor 46 and compares the current pressure with a predetermined value stored in the control system memory: if the difference between the two pressure values is less than predetermined limit and, if the flow rate is less than a predetermined minimum value also stored in the control system memory, then the control system will activate one or more of the alarms included in the device.

Referring now to FIGS. 5 to 9 which show various views and cross sections of a preferred embodiment of apparatus 200 embodying the present invention. The preferred embodiment is of generally oval shape in plan and comprises a device unit 202 and a waste canister 204 connected together by catch arrangements 206. The device unit 202 has a liquid crystal display (LCD) 208, which gives text based feedback on the wound therapy being applied, and a membrane keypad 210, the LCD being visible through the membrane of the keypad to enable a user to adjust or set the therapy to be applied to the wound (not shown). The device has a lower, generally transverse face 212 in the centre of which is a spigot 214 which forms the suction/entry port 216 to which the aspiration means (to be described below) are connected within the device unit. The lower edge of the device unit is provided with a rebated peripheral male mating face 218 which engages with a co-operating peripheral female formation 220 on an upper edge of the waste canister 204 (see FIGS. 8 and 9). On each side of the device 202, clips 222 hinged to the canister 204 have an engaging finger (not shown) which co-operates with formations in recesses 226 in the body of the device unit. From FIG. 7 it may be seen that the casing 230 of the device unit is of largely “clamshell” construction comprising front and back mouldings 232, 234, respectively and left-hand and right-hand side inserts 236, 238. Inside the casing 230 is a central chassis 240 which is fastened to an internal moulded structural member 242 and which chassis acts as a mounting for the electrical circuitry and components and also retains the battery pack 246 and aspiration pump unit 248. Various tubing items 250, 252, 254 connect the pump unit 248 and suction/entry port 216 to a final gaseous exhaust via a filter 290. FIG. 8 shows a partially sectioned side elevation of the apparatus 200, the partial section being around the junction between the device unit 202 and the waste canister 204, a cross section of which is shown at FIG. 9. Theses views show the rebated edge 218 of the male formation on the device unit co-operating with the female portion 220 defined by an upstanding flange 260 around the top face 262 of the waste canister 204. When the waste canister is joined to the device unit, the spigot 214 which has an “O” ring seal 264 therearound sealingly engages with a cylindrical tube portion 266 formed around an exhaust/exit port 268 in the waste canister. The spigot 214 of the device is not rigidly fixed to the device casing but is allowed to “float” or move in its location features in the casing to permit the spigot 214 and seal 264 to move to form the best seal with the bore of the cylindrical tube portion 266 on connection of the waste canister to the device unit. The waste canister 204 in FIG. 9 is shown in an upright orientation much as it would be when worn by a user. Thus, any exudate 270 would be in the bottom of the internal volume of waste receptacle portion 272. An aspiration conduit 274 is permanently affixed to an entry port spigot 278 defining an entry port 280 to receive fluid aspirated from a wound (not shown) via the conduit 274. Filter members 282 comprising a 0.2 μm filter and 284 comprising a fpm filter are located by a filter retainer moulding 286 adjacent a top closure member or bulkhead 288 the filter members preventing any liquid or bacteria from being drawn out of the exhaust exit port 268 into the pump and aspiration path through to an exhaust and filter unit 290 which is connected to a casing outlet moulding at 291 via an exhaust tube (not shown) in casing side piece 236. The side pieces 236, 238 are provided with recesses 292 having support pins 294 therein to locate a carrying strap (not shown) for use by the patient. The side pieces 230 and canister 204 are also provided with features which prevent the canister and device from exhibiting a mutual “wobble” when connected together. Ribs (not shown) extending between the canister top closure member 288 and the inner face 300 of the upstanding flange 260 locate in grooves 302 in the device sidewalls when canister and device are connected. The casing 230 also houses all of the electrical equipment and control and power management features, the functioning of which was described briefly with respect to FIGS. 3 and 4 hereinabove. The side piece 238 is provided with a socket member 298 to receive a charging jack from an external mains powered battery charger (both not shown).

Referring now to FIGS. 10 to 12 where a preferred embodiment of apparatus according to the present invention is described and the same features are denoted by common reference numerals.

Gaseous fluid exits from the waste canister 204 via spigot 214 into the gaseous fluid flow path defined in its initial stage to the pump 248 inlet port 410 by conduit 412 attached to the spigot 214. A silencing system is provided between the pump outlet port 414 and the exhaust outlet 408. A first exhaust conduit portion 420 is provided between the pump outlet port 414 and a plenum chamber 404; and, a second exhaust conduit portion 422 between the plenum chamber 404 and the exhaust outlet 408. However, the first exhaust conduit portion 420 is provided with a flow meter 424 intermediate its ends at the pump outlet 414 and an inlet 426 of the plenum chamber 404. A pressure sensing device 440 is connected to the first conduit portion 412 by means of a T-piece 442 and conduit 444. Similarly a pressure relief valve 448 is connected into the first exhaust conduit portion 412 also by a T-piece 450 and conduit 452. The pressure relief valve 448 is a safety device for preventing excessive negative pressures from being applied by the apparatus to the wound of user. The flow meter 424 is provided to fulfil various control functions including determining when the waste canister 204 is full. Since it is necessary that the fluid flow values measured by the flow meter 424 are accurate it is preferably positioned in the first conduit portion 420 rather than the second conduit portion 422 in case the plenum chamber 404 should leak for any reason and cause spurious flow measurements. However, other than this reason, the flow meter 424 could be positioned in the second conduit portion 422. The flow meter 424 and the pressure sensing device 440 are both connected electrically to the control system described hereinabove with reference to FIGS. 3 and 4. The second exhaust conduit portion 422 is connected to an outlet 430 of the plenum chamber 404 and the exhaust outlet 408. At the exhaust outlet 408, the second exhaust conduit portion 422 is connected to the moulding 291 which locates in the outer casing side piece 236 described with reference to FIG. 7 hereinabove. The moulding 291 may contain a final diffuser element such as an open-pore foam pad (not shown) to further break up any remaining sound waves which reach the outlet 408, however, such a foam pad may not be employed as the first and second exhaust conduit portions and the plenum chamber are generally sufficient to reduce the exhaust noise level to an acceptably low level. The plenum chamber 404 comprises a rectangular box-like structure 436 having a lower open face 432 and inlet 426 and outlet 430 and is fixed to the moulded structural member 242. A sealing gasket 434 is provided to engage with an outer rim defining the open face 432. When the complete device 230 is assembled, the rear outer case member 234 (see FIG. 7) has an upstanding rectangular rim 438 which both receives the sealing gasket 434 therein and also the rim defining the lower open face 432 of the plenum chamber 404. Thus when the front and rear casing members 232, 234 are fixed together such as by screws (not shown), for example, the sealing gasket 43 is squeezed between the outer casing member 234 and the rim defining the open face 432 to completely seal the plenum chamber 404. Before assembly the internal volume of the plenum chamber box 436 is filled with fibrous or porous sound absorbing material (not shown) which also serves as an odour filter by virtue of being impregnated with a suitable material such as activated charcoal, for example. The sound absorbing and odour filtering material serves to break up sound waves as they bounce back and forth between opposing walls of the box-like structure 436.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. An apparatus for the provision of topical negative pressure therapy, the apparatus comprising:
an aspirant pump for moving aspirated fluid through said apparatus;
aspirant conduit means operably connected to a dressing covering a wound being aspirated;
a waste container operably connected to the aspirant conduit and for receiving wound exudate therein, the waste container having a fluid exit port for the flow of gaseous aspirated fluid therefrom, the exit port having filter means associated therewith for preventing aspirated liquid from passing therethrough;
a fluid flow path on the exit side of said waste container for the flow of aspirated gaseous fluid therethrough; the fluid flow path having therein a flow meter, a plenum chamber and a pressure monitor, said flow meter positioned intermediate the exit side of said waste container and said plenum chamber on the fluid flow path; and
a control system for receiving signals from said flow meter and pressure monitor.

2. The apparatus according to claim 1, wherein the aspirant pump is positioned intermediate the pressure monitor and the flow meter.

3. The apparatus according to claim 1, wherein the apparatus further comprises an alarm triggered by the control system in the event of a waste canister full condition.

4. The apparatus according to claim 3, wherein the alarm system includes at least one of an audible alarm, a visible alarm, a physically sensed alarm, and a message alarm on a liquid crystal display (LCD) screen.

5. (canceled)

6. (canceled)

7. (canceled)

8. (canceled)

9. An apparatus for the provision of topical negative pressure therapy, the apparatus comprising:
an aspirant pump for moving aspirated fluid through said apparatus;
an aspirant conduit operably connected to a dressing covering a wound being aspirated;
a waste container operably connected to the aspirant conduit and configured to receive wound exudate therein, the waste container having a fluid exit port for the flow of gaseous aspirated fluid therefrom, the exit port having a filter associated therewith for inhibiting aspirated liquid from passing therethrough;
a fluid flow path on the exit side of said waste container for the flow of aspirated gaseous fluid therethrough; the fluid flow path having a flow meter in communication therewith, a plenum chamber and a pressure monitor, said flow meter positioned intermediate the exit side of said waste container and said plenum chamber on the fluid flow path; and
a control system configured to receive and process signals from said flow meter and pressure monitor.

10. The apparatus according to claim 9, wherein the aspirant pump is positioned intermediate the pressure monitor and the flow meter.

11. The apparatus according to claim 9, wherein the apparatus further comprises an alarm triggered by the control system in the event of a waste canister full condition.

12. The apparatus according to claim 11, wherein the alarm system includes at least one of an audible alarm, a visible alarm, a physically sensed alarm, and a message alarm on a LCD screen.

13. A method for alerting a user of a canister status for a topical negative pressure (TNP) system comprising:
receiving a measurement of a pressure generated by a pump of a TNP system from a pressure sensor;
determining a pressure difference between the measured pressure and a reference pressure;
comparing the pressure difference to a predetermined allowable pressure difference;
receiving a measurement of a flow rate on a fluid flow path of the TNP system from a flow meter;
comparing the measured flow rate with a predetermined flow rate; and
generating an alert signal in response to the comparison of the pressure difference and the comparison of the flow rate.

14. The method according to claim 13, wherein the alert signal comprises at least one of an audible alarm, a visible alarm, a physically sensed alarm, and a message alarm on a liquid crystal display (LCD) screen.

15. The method according to claim 13, wherein the canister status comprises at least one of a canister full status, a canister near full status, and a fluid flow path blockage.

16. The method according to claim 13, wherein the predetermined flow rate is a predetermined minimum flow rate.

17. The method according to claim 16, wherein comparing the measured flow rate comprises determining whether the current flow rate is greater than the predetermined minimum flow rate.

18. The method according to claim 13, wherein comparing the pressure difference comprises determining whether the pressure difference is less than an allowable pressure limit.
